(19) United States
(12) Patent Application Publication
(10) Pub. No.: US 2021/0215690 A1
(43) Pub. Date: Jul. 15, 2021

(54) METHOD FOR IMPROVING RESULT OF MONOCLONAL ANTIBODY DETECTION

(71) Applicant: SHIMADZU CORPORATION, Kyoto (JP)

(72) Inventors: Takashi SHIMADA, Kyoto (JP); Noriko IWAMOTO, Kyoto (JP)

(73) Assignee: SHIMADZU CORPORATION, Kyoto (JP)

(21) Appl. No.: 16/967,502

(22) PCT Filed: Feb. 8, 2018
(86) PCT No.: PCT/JP2018/004425

§ 371 Date: Aug. 5, 2020

(51) Int. Cl. G01N 33/543

(52) U.S. Cl. CPC G01N 33/54393

(57) ABSTRACT

The present invention provides a method for detecting a monoclonal antibody in a sample, comprising: (a) a step of capturing the monoclonal antibody in the sample and immobilizing the monoclonal antibody in pores of a porous body; (b) a step of bringing the porous body in which the monoclonal antibody is immobilized with nanoparticles on which protease is immobilized to conduct selective protease digestion of the monoclonal antibody; and (c) a step of detecting, by a liquid chromatography mass spectrometry (LC-MS), peptide fragments obtained by the selective protease digestion, wherein the selective protease digestion of step (b) is conducted at pH 8 to 9 in the presence of a chaotropic reagent and a reducing agent.

### TECHNICAL FIELD

The present invention relates to a method for improving detection results in quantification of monoclonal antibody using mass spectrometry. Still more specifically, the present invention relates to improvement of a protocol that has been established for quantification of a monoclonal antibody.

### BACKGROUND ART

Recently, intensive efforts have been made to develop bio-analysis of antibody drugs by using the LC-MS/MS technique as a quantification method in replacement of the ELISA technique.

The group of the present inventors have found that protease digestion of a monoclonal antibody by a site-selective solid phase-solid phase reaction is possible by immobilizing both of the monoclonal antibody to be measured and a protease capable of digesting the monoclonal antibody as a substrate onto a solid phase, thereby successfully obtaining peptides specific to individual monoclonal antibodies (see Patent Literatures 1 to 6, and Non Patent Literatures 1 to 8). This method is a pretreatment method for mass spectrometry in which selective protease digestion of a monoclonal antibody is carried out in such a manner that a porous body having the monoclonal antibody immobilized in pores thereof is brought into contact with nanoparticles having a protease immobilized thereon in a liquid, and is a groundbreaking technology that allows effective detection and quantification of obtained peptide fragments by liquid chromatography mass spectrometry (LC-MS). The present inventors named this method as “nano-surface and molecular-orientation limited proteolysis method (nSMOL method)”.

Quantification of an antibody drug in blood by the nSMOL method is a method that carries out trypsin digestion selectively digesting only the Fab region having a sequence specific to the antibody drug and that inhibits the ion suppression effect most problematic in the LC-MS/MS analysis, thereby making it possible to provide more stable and highly reliable quantification values. The present inventors have already confirmed that a method for detecting a monoclonal antibody using the combination of the nSMOL method and the LC-MS/MS method meets the standards of the guidelines for validation of biological analysis methods in Japan, the United States, and Europe, in terms of measuring blood levels of 15 or more kinds of antibody drugs.

On the other hand, it is known that proteins, which are biopolymers, include some proteins with a structure and a site that are very characteristically rigid. For example, it is known that the effects of amyloid beta, transferrin, and multiple transmembrane proteins (such as rhodopsin and transporter) are controlled by the presence of a rigid structure, even though the mechanisms of the controls are different.

One of such a structure to keep the rigidity of proteins is a cystine knot structure in which a knot-like structure is formed by an SS bond. Examples of molecules having the cystine knot structure and contributing to specific signal transduction include vascular endothelial growth factor (VEGF) and cytokines such as interleukins. Similar knot-like structures can be found in extracellular domains of cytokine receptors such as tumor necrosis factor (TNF) receptor. On the other hand, it is known that thioredoxin, lactoglobulin, insulin, trypsin inhibitor, haptoglobin, alpha-1 acid glycoprotein, and the like have some protease tolerance even without very strong SS bond.

Antibody molecules are high-molecular weight tetramer proteins having two heavy chains and two light chains, each of which has a specific amino acid sequence with variable regions defining the diversity and function of the antibody and constant regions having the same molecular structure. In the variable regions, complementarity determining regions (CDR) are regions in which frequency of mutation is especially high, thereby determining the binding property with antigens. Moreover, between the CH1 domain and the CH2 domain in the heavy chain constant region, there is a structure called a hinge, which has a very high flexibility.

The presence of a hinge in antibody molecules secures 3-dimensional fluctuation of the antibody binding site (fragment antigen binding, Fab). Molecular dynamics analysis such as nuclear magnetic resonance (NMR) analysis has shown that the Fc is almost constant 3-dimensionally but Fab is so largely fluctuated structurally that Fab cannot be 3-dimensionally assigned. Once an antigen binds to Fab, the fluctuation disappears, thereby converting Fab into a rigid structure, which was demonstrated also by 3-dimensional structural analysis and crystal structural analysis of the complex.

### CITATION LIST

### Patent Literatures

Patent Literature 1: International Publication No. 2015/033479
Patent Literature 2: International Publication No. 2016/194114
Patent Literature 3: International Publication No. 2016/143224
Patent Literature 4: International Publication No. 2016/143223
Patent Literature 5: International Publication No. 2016/143226
Patent Literature 6: International Publication No. 2016/143227

### Non Patent Literature

Non Patent Literature 1: Analyst. 2014 Feb. 7; 139(3):576-80. doi:10.1039/c3an02104a
Non Patent Literature 2: Anal. Methods, 2015; 21:9177-9183. doi:10.1039/c5ay01588j
Non Patent Literature 3: Drug Metabolism and Pharmacokinetics, 2016; 31:46-50. doi:10.1016/j.dmpk.2015.11.004
Non Patent Literature 4: Bioanalysis. 2016; 8(10):1009-20. doi:10.4155. bio-2016-0018
Non Patent Literature 5: Biol Pharm Bull, 2016; 39(7):1187-94. doi:10.1248/bpb.b16-00230
Non Patent Literature 6: J Chromatogr B Analyt Technol Biomed Life Sci; 2016; 1023-1024:9-16. doi:10.1016/j.jchromb.2016.04.038
Non Patent Literature 7: Clin Pharmacol Biopharm 2016; 5:164. doi:10.4172/2167-065X.1000164
Non Patent Literature 8: J. Pharm Biomed Anal; 2017; 145:33-39. doi:10.1016/j.jpba.2017.06.032

### SUMMARY OF INVENTION

### Technical Problem

The nSMOL method is based on a reaction mechanism in which a protease immobilized on the solid surface of nanoparticles of about 200 nm in diameter is brought into contact with immunoglobulin molecules immobilized on the porous body with pore diameter of about 100 nm, so that Fab of the immunoglobulin molecules is selectively cleaved in restricted reaction field. The nSMOL method is excellent in accuracy, sensitivity, and reproducibility. For performing the nSMOL method, the “nSMOL Antibody BA kit” (Shimadzu Corporation), which is a pretreatment kit for LC/MS/MS, has been commercially available with a protocol, and the present inventors have diligently studied for improvement or the like of the protocol in order to further expand the versatility of the nSMOL method.

As a result of performing detection by the nSMOL method for various antibodies, the present inventors found some cases in which an antibody-specific peptide (signature peptide) could not be cleaved even when the selective digestion reaction by protease had proceeded. In fact, there are some cases where the signature peptide is not detectable even though the reaction of the nSMOL method has proceeded.

Like other types of proteins, there is a possibility that an antibody protein may have a highly rigid region in its molecule. Such an antibody molecule may tend to have tolerance against protease, which may be result in limited degradation by the nSMOL method.

It is known that antibody molecules would have a random amino acid sequence due to class switching, somatic mutation, or the like. Moreover, even if the amino acid sequence of an antibody is known, structural prediction is extremely difficult especially for the variable region thereof. Therefore, it is practically impossible to predict which conditions are optimum for which antibody in performing the antibody detection using the nSMOL method.

An object of the present invention is to propose analysis conditions for rigid monoclonal antibodies as described above in order to make the nSMOL method applicable to any monoclonal antibody drugs, thereby further expanding the versatility of the nSMOL method.

### Solution to Problem

While not wishing to be bound by any theory, the present inventors predicted a possibility that the low detection result would be caused by the protease tolerance derived from the rigidity of the antibody molecule to be analyzed. That is, the present inventors deduced a possibility that there would be a highly rigid region in such an antibody molecule due to some mechanism, thereby making the antibody molecule tolerant against protease, as a result of which the protease digestion expected in the nSMOL method will not proceed.

The nSMOL method is, by theory, configured to perform 3-dimensional structural control of contact between protease and a substrate, and therefore it is assumed that the protease reaction selectively proceeds for Fab regions of any antibody. It has been already proved that a reaction independent on the diversity of antibodies surely proceeds. However, in case where the antibody molecule itself is highly rigid, there is a possibility that hydrolysis, which allows antibody quantification, would not proceed even if protease is brought into contact with the substrate.

The present inventors have studied various analysis conditions for such rigid monoclonal antibodies in order to apply the nSMOL method for any monoclonal antibody drugs. As a result, the present inventors found that detection results are significantly improved by performing the selective protease digestion of the monoclonal antibody by contacting it with protease in the presence of a chaotropic reagent and a reducing agent. While not wishing to be bound by any theory, it is deduced that this effect is obtained by relaxing the rigid 3-dimensional structure of the antibody thereby facilitating the protease digestion reaction, and improving the releasing efficiency of peptides released as a result of the protease digestion.

That is, the present invention provides the followings.

1. A method for improving a detection sensitivity in a detection method for a monoclonal antibody in a sample, the detection method including:

(a) a step of capturing the monoclonal antibody in the sample and immobilizing the monoclonal antibody in pores of a porous body;

(b) a step of bringing the porous body in which the monoclonal antibody is immobilized with nanoparticles on which protease is immobilized to conduct selective protease digestion of the monoclonal antibody; and

(c) a step of detecting, by a liquid chromatography mass spectrometry (LC-MS), peptide fragments obtained by the selective protease digestion,

wherein the selective protease digestion of step (b) is conducted at pH 8 to 9 in the presence of a chaotropic reagent and a reducing agent.

2. The method according to 1 above, wherein the chaotropic reagent is selected from the group consisting of guanidinium hydrochloride, urea, thiourea, ethylene glycol, and ammonium sulfate.
3. The method according to 2 above, wherein the chaotropic reagent is urea or thiourea in the concentration range of 0.5 to 3 M.
4. The method according to any one of 1 to 3 above, wherein the reducing agent is selected from the group consisting of dithiothreitol (DTT), tris(2-carboxyethyl)phosphine (TCEP) or a hydrochloride salt thereof, and tributyl phosphine.
5. The method according to 4 above, wherein the reducing agent is TCEP in the concentration range of 0.1 to 0.5 mM.
6. Use of a chaotropic reagent and a reducing agent for improving detection sensitivity in a detection method of a monoclonal antibody in a sample, the detection method comprising:
(a) a step of capturing the monoclonal antibody in the sample and immobilizing the monoclonal antibody in pores of a porous body;
(b) a step of bringing the porous body in which the monoclonal antibody is immobilized with nanoparticles on which protease is immobilized to conduct selective protease digestion of the monoclonal antibody; and
(c) a step of detecting, by a liquid chromatography mass spectrometry (LC-MS), peptide fragments obtained by the selective protease digestion,
   7. The use according to 6 above, wherein the chaotropic reagent is selected from the group consisting of guanidinium hydrochloride, urea, thiourea, ethylene glycol, and ammonium sulfate.
   8. The use according to 7 above, wherein the chaotropic reagent is urea or thiourea in the concentration range of 0.5 to 3 M.
   9. The use according to any one of 6 to 8 above, wherein the reducing agent is selected from the group consisting of dithiothreitol (DTT), tris(2-carboxyethyl)phosphine (TCEP) or a hydrochloride salt thereof, and tributyl phosphine.
   10. The use according to 9 above, wherein the reducing agent is TCEP in the concentration range of 0.1 to 0.5 mM.

### Advantageous Effects of Invention

According to the present invention, a quantification method for which analysis validation is possible is established for monoclonal antibodies considered as having a rigid chemical structure, such as Adalimumab and Trastuzumab, thereby making it possible to apply the nSMOL method to a wider range of antibodies than the conventional nSMOL method. The method according to the present invention not only improves the detection sensitivity for any antibodies, but also makes it possible to detect a lower concentration of antibodies. Thus, according to the present invention, it is possible to provide a protocol of the nSMOL method with a greater versatility.

### BRIEF DESCRIPTION OF DRAWINGS

FIG. 1 illustrates amino acid sequences of a heavy chain Fab domain (left) and a light chain (right) of Adalimumab. The underlined portion indicates a peptide (SEQ ID NO: 3) used as a signature peptide.

FIG. 2 illustrates peak intensities of a signature peptide detected when the nSMOL method was performed at pH8, pH8.5, or pH9 and ratios thereof against a peak intensity of P14R used as an internal standard (ISTD ratio), where the “sum” indicates the peak intensity of the signature peptide.

FIG. 3 illustrates results of comparisons of peak intensities and ISTD ratios of the signature peptide detected when the nSMOL method was performed at pH8, pH8.5, and pH9 with 2 mM of TCEP as a reducing agent and with and without 1 M of urea.

FIG. 4 illustrates peak intensities and ISTD ratios of the signature peptide detected when the nSMOL method was performed with 1 M of urea as a chaotropic reagent in the presence of TCEP in the range of 0.5 to 3 mM.

FIG. 5 illustrates peak intensities and ISTD ratios of the signature peptide detected when the nSMOL method was performed with 2 M of urea as a chaotropic reagent and without TCEP or with TCEP in the range of 0.1 to 0.3 mM.

FIG. 6 illustrates peak intensities and ISTD ratios of the signature peptide detected when the nSMOL method was performed with 2 M of urea as a chaotropic reagent in the presence of TCEP in the range of 0.01 to 0.2 mM.

FIG. 7 illustrates peak intensities and ISTD ratios of the signature peptide detected when the nSMOL method was performed with 0.5 mM TCEP as a reducing agent without urea or with 1 M or 2 M urea.

FIG. 8 illustrates peak intensities and ISTD ratios of the signature peptide detected when the nSMOL method was performed with 0 to 3 M of urea as a chaotropic reagent and with 0.01 to 0.2 mM of TCEP as a reducing agent.

FIG. 9 illustrates peak intensity and an ISTD ratio of the signature peptide detected when the nSMOL method was performed at pH 8.5 with 2 M urea and 0.2 mM TCEP (Urea/TCEP, right), compared with those obtained at pH 8 without the chaotropic reagent and the reducing agent (control, left).

FIG. 10 illustrates plotting of results of detection of 2 to 250 μg/ml of Adalimumab in samples at pH 8.5 with 2M urea and 0.2 mM TCEP against the horizontal axis of ratio of set concentrations over detected concentrations and the vertical axis of the ratio of the peak intensities (areas) of the signature peptide over the ISTD.

FIG. 11 illustrates results of comparison of peak intensities of Trastuzumab, Cetuximab, Rituximab, and Nivolumab, detected at pH 8.5 with 2M urea in the presence of 0.1, 0.2, or 0.5 mM TCEP, showing the peak intensities as relative intensities to the highest peak intensity (obtained in the three TCEP concentrations set to 1 for each monoclonal antibody).

FIG. 12A illustrates peak intensities of signature peptides of Trastuzumab, Cetuximab, Rituximab, and Nivolumab, when the nSMOL method was performed at pH 8.5 with 2M urea and 0.2 mM TCEP (right), in comparison with those obtained at pH 8 without the chaotropic reagent and the reducing agent (control, left), showing the peak intensities as relative intensities to the peak intensity of control set to 1. FIG. 12B illustrates enlargements of the results of Cetuximab, Rituximab, and Nivolumab in FIG. 12A.

FIG. 13A illustrates plotting of results of detections of 0.061 to 250 μg/ml of Trastuzumab in samples at pH 8 without a chaotropic reagent and a reducing agent (♦) and at pH 8.5 with 2M urea and 0.2 mM TCEP (▪), against the horizontal axis of concentrations and the vertical axis of peak intensities. FIG. 13B illustrates enlargement of the results in the low concentration region (Trastuzumab concentration of 2.5 μg/ml or less) in FIG. 13A.

### DESCRIPTION OF EMBODIMENTS

The present invention provides a method for improving a detection sensitivity in a detection method for a monoclonal antibody in a sample, the detection method comprising:

(a) a step of capturing the monoclonal antibody in the sample and immobilizing the monoclonal antibody in pores of a porous body;

(b) a step of bringing the porous body in which the monoclonal antibody is immobilized with nanoparticles on which protease is immobilized to conduct selective protease digestion of the monoclonal antibody; and

(c) a step of detecting, by a liquid chromatography mass spectrometry (LC-MS), peptide fragments obtained by the selective protease digestion,

wherein the selective protease digestion of step (b) is conducted at pH 8 to 9 in the presence of a chaotropic reagent and a reducing agent.

The present invention also provides use of a chaotropic reagent and a reducing agent for improving detection sensitivity in a detection method of a monoclonal antibody in a sample, the detection method comprising:

(a) a step of capturing the monoclonal antibody in the sample and immobilizing the monoclonal antibody in pores of a porous body;

(b) a step of bringing the porous body in which the monoclonal antibody is immobilized with nanoparticles on which protease is immobilized to conduct selective protease digestion of the monoclonal antibody; and

(c) a step of detecting, by a liquid chromatography mass spectrometry (LC-MS), peptide fragments obtained by the selective protease digestion.

<Step (a)>

Step (a) of the method according to the present invention is a step of capturing and immobilizing, in pores of a porous body, the monoclonal antibody in the sample.

The term “sample” used herein means a liquid sample in which the presence of a monoclonal antibody is to be detected, and is not particularly limited. In general, the sample is a biological sample derived from a mammal such as a mouse, a rat, a rabbit, a goat, a bovine, a human, or the like, especially a human subject, or mainly a human patient, and preferably plasma, serum, or a tissue homogenate extract. Alternatively, the sample may be a liquid sample containing a monoclonal antibody and serum artificially added, to prove the effect of the present invention, for example. For detecting a monoclonal antibody in a method according to the present invention, the concentration of the monoclonal antibody in the sample may be in the range of 0.05 to 300 μg/ml.

Examples of the monoclonal antibody that can be a measurement target include, but not limited to, human antibodies such as Panitumumab, Ofatumumab, Golimumab, Ipilimumab, Nivolumab, Ramucirumab, Adalimumab; humanized antibodies such as Tocilizumab, Trastuzumab, Trastuzumab-DM1, Bevacizumab, Omalizumab, Mepolizumab, Gemtuzumab, Palivizumab, Ranibizumab, Certolizumab, Ocrelizumab, Mogamulizumab, Eculizumab, Tocilizumab, Mepolizumab; chimeric antibodies such as Rituximab, Cetuximab, Infliximab, Basiliximab.

Furthermore, a conjugate having an additional function added while maintaining the specificity of a monoclonal antibody, for example, Fc-fused proteins (such as etanercept, and abatacept) and antibody-drug conjugates (such as brentuximab vedotin, Gemtuzumab ozogamicin, and Trastuzumab emtansine) may also be a monoclonal antibody as a measurement target. The conjugate may be pretreated to dissociate its bonding prior to the measurement, so that only its antibody portion can be provided to the analysis. As an alternative, the conjugate as such may be provided to the analysis.

Information on amino acid sequences of monoclonal antibodies, etc. can be obtained from, for example, Kyoto Encyclopedia of Genes and Genomes, KEGG.

The porous body for use in the method according to the present invention may be a material having a large number of pores and being capable of binding with an antibody site-specifically. The average pore diameter of the porous body is approximately in the range of 10 nm to 200 nm, and is set as appropriate to be smaller than the average particle diameter of the nanoparticles.

In step (a) in the present invention, a monoclonal antibody as a measurement target is immobilized in pores of a porous body. For this purpose, a porous body, in pores of which linker molecules interactive with the antibody site-specifically are immobilized, may be preferably used.

The linker molecules may be preferably Protein A, Protein G, or the like, capable of site-specifically binding with the Fc domain of the antibody. The use of a porous body with such linker molecules immobilized in the pores thereof allows the Fc domain of the antibody to be anchored in the pores in such a way that the Fab domain is located near the surface layer in the pores, thereby allowing site-selective digestion of the Fab domain by the protease.

A porous body that can be suitably used in the present invention is not particularly limited. For example, Protein G Ultralink resin (manufactured by Pierce Corporation), Toyopearl TSKgel (manufactured by TOSOH Corporation), Toyopearl AF-rProtein A HC-650F resin (manufactured by TOSOH Corporation), Protein A Sepharose (GE Healthcare), KanCapA (KANEKA), and the like can be used.

A method for immobilizing an antibody in pores of a porous body is not particularly limited. For example, when an antibody is immobilized in a porous body in which Protein A or Protein G is immobilized in pores in advance, the antibody can be easily immobilized in pores by mixing a suspension of the porous body with a solution containing the antibody. A quantitative ratio of the porous body to the antibody can be appropriately set according to a purpose.

<Step (b)>

Step (b) of the method according to the present invention is a step of carrying out selective protease digestion of the monoclonal antibody by contacting the porous body with nanoparticles, the porous body being obtained in step (a) to have the monoclonal antibody immobilized thereon, and the nanoparticles having a protease immobilized thereon.

The protease to be immobilized on nanoparticles may be appropriately selected depending on the monoclonal antibody to be quantified or identified by mass spectrometry, and is not limited. Examples of the protease include trypsin, chymotrypsin, lysyl endopeptidase, V8 protease, Asp N protease (Asp-N), Arg C protease (Arg-C), papain, pepsin, dipeptidyl peptidase used alone or in combination. As the protease, trypsin is particularly preferably used. Examples of the protease that can be suitably used in the method of the present invention include Trypsin Gold (manufactured by Promega Corporation), Trypsin TPCK-Treated (manufactured by Sigma Corporation), and the like.

The nanoparticles have a larger average particle size than the average pore diameter of the porous body. The shape of the nanoparticles are not particularly limited. However, from a point of view of uniform access of the protease to the pores of the porous body, spherical nanoparticles are preferred. Further, it is preferable that the nanoparticles have high dispersibility and a uniform particle size.

As a type of the nanoparticles, magnetic nanoparticles that can be dispersed or suspended in an aqueous medium and can be easily recovered from the dispersion or suspension by magnetic separation or magnetic precipitation separation are preferable. Further, from a point of view that aggregation is less likely to occur, magnetic nanoparticles coated with an organic polymer are more preferable. Specific examples of magnetic nanobeads coated with an organic polymer include FG beads. SG beads, Adembeads, nanomag, and the like. As a commercially available product, for example, FG beads (polymer magnetic nanoparticles having a particle size of about 200 nm obtained by coating ferrite particles with polyglycidyl methacrylate (poly GMA)) manufactured by Tamagawa Seiki Co., Ltd. are suitably used.

In order to suppress nonspecific protein adsorption and to selectively immobilize the protease, the nanoparticles are preferably modified with spacer molecules capable of binding to the protease. By immobilizing a protease via a spacer molecule, desorption of the protease from surfaces of the nanoparticles is suppressed, and regioselectivity of protease digestion is improved. Further, by adjusting the molecular size of a spacer, a protease can selectively access a desired position of an antibody, and thus site-selectivity can be improved.

Nanoparticles surface-modified with such spacer molecules are also commercially available, for example, nanoparticles modified with a spacer molecule having an ester group activated with N-hydroxysuccinimide (active ester group) are commercially available under a trade name of “FG beads NHS” (Tamagawa Seiki Co., Ltd.).

A method for immobilizing a protease on surfaces of nanoparticles is not particularly limited. An appropriate method can be adopted according to characteristics of the protease and the nanoparticles (or spacer molecules modifying the surfaces of the nanoparticles). The aforementioned pretreatment kit for LC/MS/MS “nSMOL Antibody BA Kit” (Shimadzu Corporation) includes “FG beads Trypsin DART®”, nanoparticles on which trypsin is immobilized as a protease, which can suitably be used for the method of the present invention.

By contacting the porous body having the monoclonal antibody immobilized thereon with the nanoparticles having the protease immobilized thereon, the selective protease digestion of the monoclonal antibody is achieved, thereby producing peptide fragments.

The protease digestion may be, for example, conducted in a buffer solution having pH adjusted to the optimum pH for protease or a vicinity thereof. For the purpose of the present invention, it is preferable that the protease digestion be conducted at pH within the range of pH 8 to 9, or especially at about pH 8.5. The reaction temperature for the protease digestion may be at about 37° C., but it is preferable to carry out the protease digestion at about 50° C. under saturated vapor pressure. The reaction time may be in the range of 30 min to 20 hours, for example, 1 hour to 8 hours, or 3 hours to 5 hours. It is preferable that the reaction be maintained under saturated vapor pressure in order to prevent evaporation of the reaction solution, but the present invention is not limited to this configuration.

Step (b) may be configured to include stirring of the reaction solution, so as to facilitate the contact of the porous body with the nanoparticles. The stirring may be conducted over the whole reaction time, or only during part of the reaction time such as only during a reaction initial stage, and therefore the stirring is not limited to particular duration and timing.

In the present invention, the protease digestion in step (b) is conducted in the presence of a chaotropic reagent and a reducing agent.

The chaotropic reagent can be selected from, but not limited to, the group consisting of, for example, guanidinium hydrochloride, urea, thiourea, ethylene glycol, and ammonium sulfate. Among these, the chaotropic reagent may be preferably urea or thiourea, or especially urea, because the chaotropic reagent should preferably not cause adverse effects such as damaging resin in a column used in LC-MS in step (c) described below, and not affect pH.

When using urea or thiourea, it is preferable that the concentration thereof in the reaction of step (b) be within the range of 0.5 to 2 M, especially within the range of 1 to 2 M. If using a concentration exceeding about 6 M, this would denature the antibody protein, thereby rather deteriorating the detection effect. Therefore, the ranges of concentrations mentioned above are well lower than the concentration of urea used as a denaturing agent for proteins.

The reducing agent may be, but not limited to, one selected from the group consisting of dithiothreitol (DTT), tris(2-carboxyethyle)phosphine (TCEP), or a hydrochloride salt thereof, and tributyl phosphine, for example. These reducing agents are available from Sigma-Aldrich Co. LLC, NACALAI TESQUE, INC., Funakoshi Co., Ltd., and other suppliers. Preferably, the reducing agent may be TCEP that exhibits a good reducing capacity in a wide pH range.

In case where TCEP is used, the TCEP concentration in the reaction in step (b) may be preferably in the range of 0.05 to 1 mM, especially in the range of 0.1 to 0.5 mM, such as 0.1 mM, 0.15 mM, 0.2 mM, 0.25 mM, 0.3 mM, 0.35 mM, 0.4 mM, 0.45 mM, or 0.5 mM. These concentration ranges are well lower than ordinary concentrations adopted when using TCEP as a reducing agent for allowing complete cleavage of SS bonds existing in a reaction.

As described above, very surprisingly, the optimum concentration ranges of the chaotropic reagent and the reducing agent for achieving the effects of the present invention are significantly lower than the concentrations usually used in this field. It is deduced that the presence of both the chaotropic reagent and the reducing agent with such low concentrations facilitates the bringing the antibody to be substrate into contact with the protease on the nanoparticle surface, improves the stability of the peptides cleaved and released, and probably prevents the peptides from being adsorbed onto the vessel or being oxidized by contact with air, thereby contributing to the improvement of detection sensitivity.

Peptides obtained by the protease digestion described above are dissolved and released in the reaction solution. Therefore, in order to subject a target peptide fragment to mass spectrometry, it is necessary to remove the porous body and the nanoparticles. This can be achieved by subjecting the sample after the protease digestion to filtration, centrifugation, magnetic separation, dialysis, and the like.

For example, by filtration using a filtration membrane made of polyvinylidene fluoride (PVDF) (such as low-binding hydrophilic PVDF having a pore diameter of 0.2 μm manufactured by Millipore Corporation), a filtration membrane made of polytetrafluoroethylene (PTFE) (such as low-binding hydrophilic PTFE having a pore diameter of 0.2 μm manufactured by Millipore Corporation), and the like, the porous body and the nanoparticles can be easily removed. The filtration may be centrifugal filtration, which allows prompt and easy filtration.

As described above, TCEP is preferable as the reducing agent because, when the TCEP is selected, it is not quite probable that a small amount of the reducing agent remaining at the end of step (b) would hinder later operation, and the improvement in the stability of peptides is expected.

<Step (c)>

Step (c) of the method according to the present invention is a step of detecting, by using liquid chromatography mass spectrometry (LC-MS), peptide fragments obtained by the selective protease digestion.

An ionization method in mass spectrometry and an analysis method of ionized sample are not particularly limited. Further, MS/MS analysis, multistage mass spectrometry of MS3 or higher, or multiple reaction monitoring (MRM) can also be performed using a triple quadrupole mass spectrometer or the like.

Examples of an apparatus especially suitable for the method of the present invention include, but not limited to, LCMS-8030, LCMS-8040, LCMS-8050, LCMS-8060 (all from Shimadzu Corporation), and LCMS-IT-TOF (Shimadzu Corporation).

With the mass spectrometry or the like, a peptide fragment including an amino acid sequence of a Fab region specific to a target monoclonal antibody, for example, CDR1 region, CDR2 region, and/or CDR3 region of a heavy chain and/or a light chain can be detected, and it is possible to identify and/or quantify the target monoclonal antibody.

Amino acid sequence information etc. of monoclonal antibodies intended to be used as an antibody drug have been published, so that information of amino acid sequences of heavy chains and light chains, Fab and Fc domains, complementarity determining regions (CDRs), disulphide bond, etc. are available. The protease digestion according to the nSMOL method produces a plurality of peptides, and amino acid sequence information of each of the peptides is available. Accordingly, it can be easily understood at which position in the monoclonal antibody the peptide locates. Therefore, it is possible to select an especially suitable peptide as an analysis target from among a plurality of peptides derived from Fab regions. Such a peptide thus selected is called a “signature peptide”.

Details of nSMOL method are disclosed, for example, in WO2015/033479; WO2016/143223; WO2016/143224; WO2016/143226; WO2016/143227; WO2016/194114; Analyst. 2014 Feb. 7; 139(3): 576-80, doi: 10.1039/c3an02104a; Anal. Methods, 2015; 21: 9177-9183. doi:10.1039/c5ay01588j; Drug Metabolism and Pharmacokinetics, 2016; 31: 46-50. doi:10.1016/j.dmpk.2015.11.004; Bioanalysis. 2016; 8(10):1009-20. doi: 10.4155. bio-2016-0018; Biol Pharm Bull, 2016; 39(7):1187-94. doi: 10.1248/bpb.b16-00230; J Chromatogr B Analyt Technol Biomed Life Sci; 2016; 1023-1024:9-16. doi: 10.1016/j.jchromb.2016.04.038; Clin Pharmacol Biopharm 2016; 5:164. doi:10.4172/2167-065X.1000164; and J. Pharm Biomed Anal; 2017; 145:33-39. doi:10.1016/j.jpba.2017.06.032; and the like. The contents disclosed in these literatures are incorporated herein by reference.

One example of a conventional protocol of the nSMOL method is as below.

<Step (a)>

1. Dilute a biological sample of 5 to 10 μL containing a monoclonal antibody with PBS+0.1% n-octyl thioglycoside (OTG) in an amount that is larger than the amount of the sample by about 10 to 20 times.

2. Add 25 μL of a porous body suspension (TOYOPEARL AF-rProtein A HC-650F, 50% slurry) therein.

3. Conduct vortex stirring of the sample solution gently for about 5 min.

4. Collect the whole sample solution with ultrafree low-protein binding Durapore PFDF (0.22 μm).

5. Conduct centrifugal separation to remove supernatant (10,000 g×1 min).

6. Add 300 μL of PBS+0.1% OTG therein, and conduct centrifugal separation to remove supernatant (10,000 g×1 min).

7. Repeat step 6.

8. For removing the surfactant, add 300 μL of PBS therein, and conduct centrifugal separation to remove supernatant (10.000 g×1 min).

9. Repeat step 8.

10. Add 75 to 100 μL of a reaction solution (25 mM Tris-HCL, pH8), in which 10 fmol/μL of P14R synthetic peptide have been added in advance.

<Step (b)>

11. Add 5 to 10 μL of nanoparticles on which chemically-modified trypsin is immobilized (0.5 mg/ml FG beads suspension).

12. Conduct the reaction for 4 to 6 hours, while gently stirring the reaction solution at 50° C. under saturated vapor pressure.

13. Add 10 μL of 10% formic acid to the reaction solution to terminate the reaction.

14. Centrifugate the reaction solution (10,000 g×1 min) to collect a solution.

15. Place the solution on a magnetic stand for let the solution stand for about 1 to 2 min, thereby removing excess resin.

<Step (c)>

16. Conduct LCMS analysis of the solution.

The method according to the present invention may use Tris-HCL containing a chaotropic reagent and a reducing agent as the reaction solution in “10.” in the protocol above, instead of Tris-HCL, pH8. Note that Tris-HCL is a buffer agent generally used in this field, and a similar reaction can be conducted with another buffer agent such as PBS, Bis-Tris, Tricine, Bicine, HEPES, CAPS, MES, MOPS, phosphate buffer solution, or the like, and thus the present invention is not particularly limited in terms of buffer agents.

Adalimumab, which is described herein as one example of the monoclonal antibodies having a rigid structure, is a human monoclonal antibody that can bind specifically with TNF-α, and is commercially available under the product name “Humira.”

As described above, the method according to the present invention is especially advantageous for the nSMOL method-based detection of a monoclonal antibody with a rigid structure, but may be employed for any types of monoclonal antibodies. However, it is recommendable to select the method according to the present invention, for example, in case where the detection results by the conventional nSMOL method are significantly lower than expected results, or in case where the Fab region of the monoclonal antibody is expected to contain a rigid structure.

The method according to the present invention is applicable to, for example, but not limited to, monoclonal antibodies that are difficult to be detected at a concentration of 0.5 μg/mL or less, 1 μg/mL or less, 5 μg/mL or less, or 10 μg/mL or less with the conventional nSMOL method, therefore not detectable within a concentration range sufficiently lower than the quantifiable range expected from results of pharmacokinetic studies, but also monoclonal antibodies that are detectable within such a concentration range.

Concrete examples of the monoclonal antibodies to which the method according to the present invention is suitably applicable include, but not limited to, Adalimumab, Trastuzumab, Cetuximab, Rituximab, and Nivolumab, which are described above and also in Example below.

The method according to the present invention can improve the detection sensitivity of the nSMOL method by about 2 to 100 times depending on the type of the antibodies, and improve the lower limits of the detectable range and quantifiable range by about 3 to 30 times.

### EXAMPLES

The present invention will be described in more detail, referring to Examples below. The data below are only part of the data obtained through a number of experiments, and the present invention is not limited to these Examples.

### Example 1 pH Dependency of Adalimumab Detection

Using Adalimumab as the target of measurement, improvement of the protocol for the detection of Adalimumab in a sample by the nSMOL method was studied.

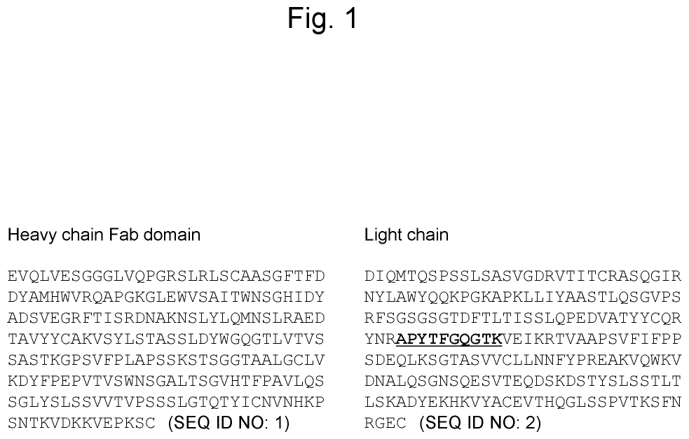

FIG. 1 illustrates amino acid sequences of variable regions of a heavy chain and a light chain of Adalimumab (SEQ ID NO: 1 and 2). For the detection of Adalimumab, by excluding a peptide which has the same sequence as a peptide derived from other antibodies which may exist in human plasma, and the like, among a plurality of peptide candidates detectable by the nSMOL method, a peptide having the sequence APYTFGQGTK (SEQ ID NO: 3) underlined was selected as a signature peptide present in the Fab region of Adalimumab.

Into 12.5 μL of a porous body suspension (TOYOPEARL AF-rProtein A HC-650F, 50% slurry), 90 μL of PBS was added. Into this suspension, 5 μL of a sample was added, where the sample had been prepared by adding Adalimumab (AbbVie GK.) in human blood plasma (manufactured by KOHJIN BIO Corporation, had been filtered with a 5-μm filter and then with a 0.8-μm filter) to make up 100 μg/mL, and the suspension was stirred gently for about 5 min.

The suspension thus prepared was transferred into a filter cup (Ultrafree MC-GV, manufactured by Millipore), and centrifuged (10,000 g×1 min) to remove the supernatant.

After the centrifugation (10,000 g×1 min) to remove the supernatant, a process including adding 300 μL of PBS containing 0.1% octyl thioglycoside and centrifuging the suspension was repeated 3 times. Then, a process including adding 300 μL of PBS and centrifuging the suspension was repeated 3 times.

As reaction solutions, solutions were prepared at pH8, pH8.5, and pH9 (25 mM Tris-HCL). Into the sample, 80 μL of one of the reaction solutions was added and then 5 μL of nanoparticles on which protease was immobilized (FG beads Trypsin DART) were added. After that, reaction was conducted at 50° C. under saturated vapor pressure for 5 hours.

After adding 5 μL of a reaction terminating solution (10% formic acid), the sample was subjected to centrifugal filtration, and a solution was collected by magnetic separation.

Using NexeraX2 system (Shimadzu Corporation) and LCMS8050 (Shimadzu Corporation), LCMS analysis was conducted.

The measurement was conducted for the peptide of SEQ ID NO: 3 mentioned above. Measurement conditions were as below.

Solvent A: 0.1% formic acid-containing aqueous solution

Solvent B: 0.1% formic acid-containing acetonitrile solution

Flow rate: 0.4 ml/min or 1 ml/min

Equilibrium concentration: % B=1.0

Column: Shimpack GISS C18, 2 mm×50 mm (Shimadzu Corporation)

Column temperature: 50° C.

HPLC Conditions:

1.50 min In-pump solvent B concentration 1%

4.70 min In-pump solvent B concentration 42%

4.71 min In-pump flow rate 0.4 ml/min

4.72 min In-pump solvent B concentration 95%

4.73 min In-pump flow rate 1 ml/min

5.65 min In-pump solvent B concentration 95%

5.66 min In-pump solvent B concentration 1%

6.05 min In-pump flow rate 1 ml/min

6.06 min In-pump flow rate 0.4 ml/min

Interface Conditions:

Nebulizer gas: 3 L/min

Heating gas: 10 L/min

Drying gas: 10 L/min

Interface temp: 300° C.

Desolvent temp: 240° C.

Heat block temp: 400° C.

Collision-Induced Dissociation Inducing Gas Conditions:

Gas used: Argon

Partial pressure used: 270 kPa

MRM Transition Conditions:

TABLE 1

| Precursor | Product | Dwell Time | Q1 voltage | Q2 voltage | Q3 voltage |
|---|---|---|---|---|---|
| m/z | m/z | [msec] | [V] | [V] | [V] |
| 534.95 | 738.40 | 10 | −26 | −23 | −28 |
| 534.95 | 901.50 | 10 | −26 | −22 | −26 |
| 534.95 | 637.40 | 10 | −26 | −23 | −24 |

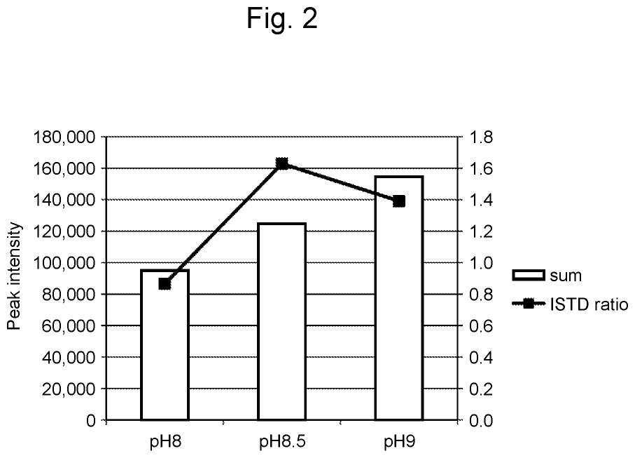

As a result, as illustrated in FIG. 2, a greater signal intensity was obtained with a higher pH. On the other hand, the ratio against P14R used as an internal standard was highest at pH 8.5 and lower at pH9. Because a high pH condition breaks down P14R and also causes random hydrolysis of the target protein, the optimum pH value was set at pH 8.5.

### Example 2 Chaotropic Reagent Dependency

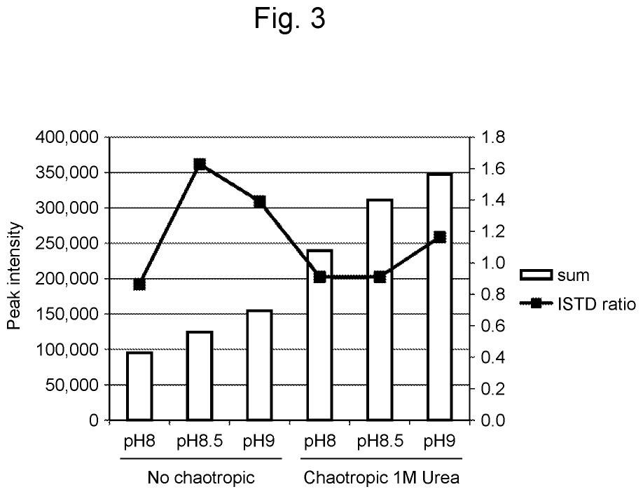

Using 2 mM TCEP (manufactured by Sigma-Aldrich Co. LLC) as a reducing agent, the effect of the presence of 1 M urea (manufactured by Sigma-Aldrich Co. LLC) as a chaotropic reagent was confirmed. The nSMOL method was conducted similarly as in Example 1 but with and without 1 M urea, and at pH 8, pH 8.5, and pH 9. As a result, as illustrated in FIG. 3, as in Example 1, it was confirmed that a greater signal intensity was obtained at a higher pH with and without the chaotropic reagent, and that the yield was higher in the presence of the chaotropic reagent.

Since the chaotropic reagent would cause denaturing effect at a high concentration, it was considered necessary to study optimum concentration of the chaotropic reagent.

### Example 3 Reducing Agent Concentration Dependency 1

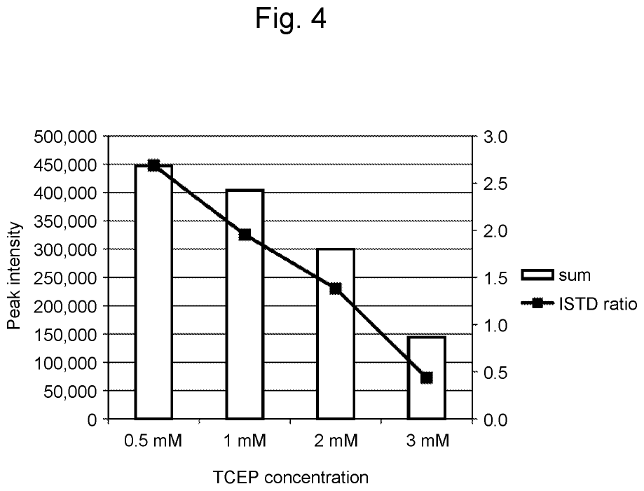

Using 1 M urea as a chaotropic reagent, the effects of different concentrations of a reducing agent was studied. More specifically, the nSMOL method was conducted similarly as in Example 1 but at pH 8.5 in the presence of TCEP in the range of 0.5 to 3 mM. As a result, as illustrated in FIG. 4, it was found that a higher reaction yield and a higher ratio to internal standard were obtained at a lower reducing agent concentration.

Therefore, it was considered necessary to study the use of the reducing agent at a lower concentration.

### Example 4 Reducing Agent Concentration Dependency 2

Using 2 M urea as a chaotropic reagent, the nSMOL method was conducted similarly as in Example 1 but at pH 8.5 in the absence of TCEP and in the presence of TCEP in the range of 0.1 to 0.3 mM, which were lower concentrations than that in Example 3.

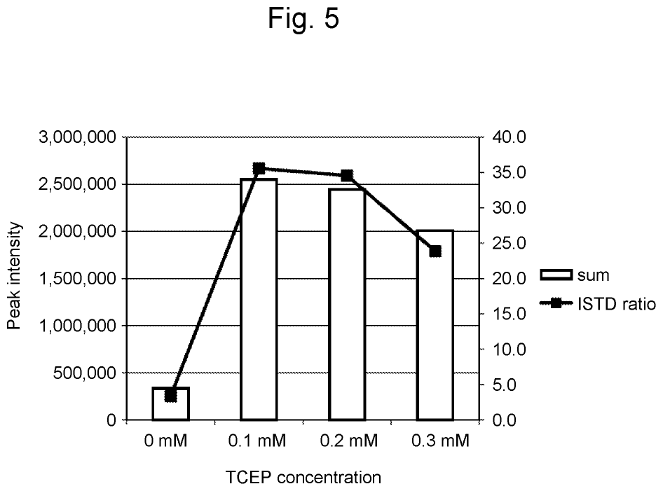

As a result, as illustrated in FIG. 5, the peak intensity was lower in the absence of the reducing agent, whereas the peak intensity was higher with the reducing agent in the range of 0.1 to 0.3 mM. Therefore, it was demonstrated that the presence of TCEP in the range of 0.1 to 0.2 mM is optimum for the detection of Adalimumab in case where the reaction was conducted at pH 8.5 with 2 M urea.

This result confirmed that the method according to the present invention can achieve a greater yield with such a low concentration of the reducing agent, that is about 1/50 of the general concentrations of the reducing agent used in the range of 5 to 10 mM.

### Example 5 Reducing Agent Concentration Dependency 3

Using 2 M urea as a chaotropic reagent, the nSMOL method was conducted similarly as in Example 1 but at pH 8.5 in the presence of TCEP in the range of 0.01 to 0.2 mM, which were further lower concentrations than those in Example 4.

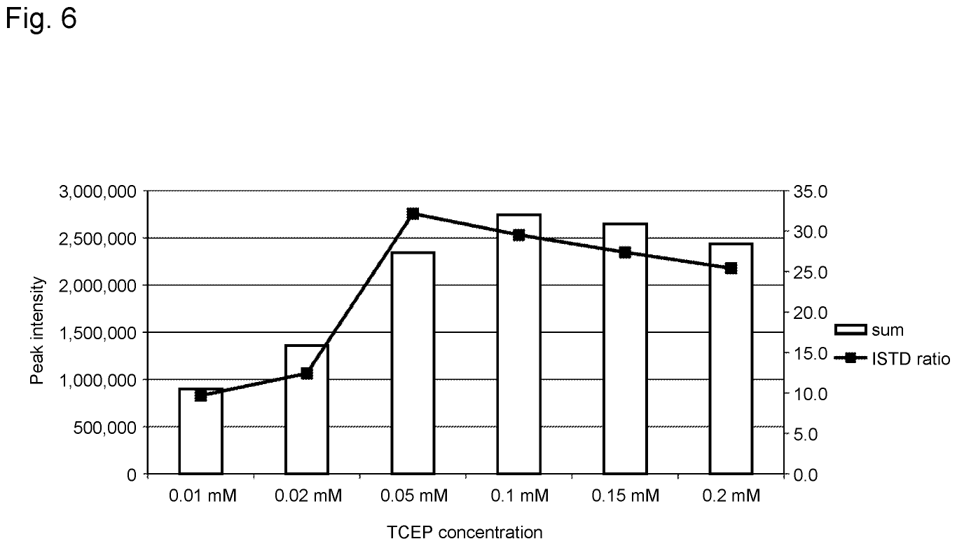

As a result, as illustrated in FIG. 6, it was confirmed that the use of TCEP was optimum at the concentration in the range of 0.05 to 0.2 mM, especially in the range of 0.1 to 0.2 mM for the detection of Adalimumab.

### Example 6 Chaotropic Reagent Concentration Dependency

Using 0.5 mM TCEP as a reducing agent, the nSMOL method was conducted similarly as in Example 1 but in the absence of urea or in the presence of 1 M or 2 M urea (at pH 8.5).

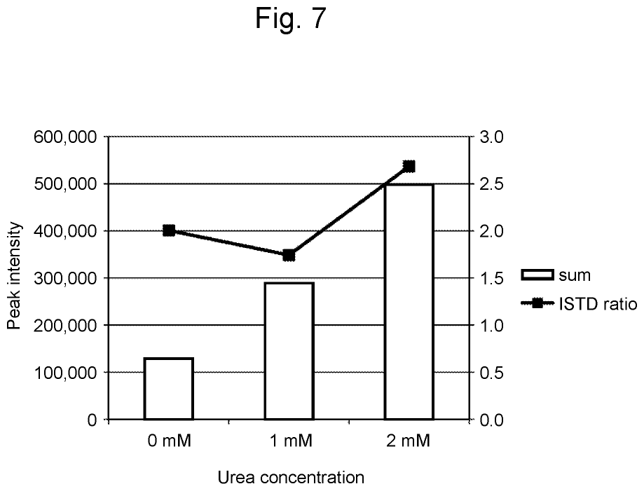

As a result, as illustrated in FIG. 7, it was confirmed that 2M urea is optimum as the chaotropic reagent in the low-concentration reducing agent usage conditions employed in the present invention. Because the protein denaturing effect would occur with about 7 M of urea in general, it was deduced that this concentration would contribute, for example, to the stabilization of free peptide, releasing efficiency, etc., but not to the denaturing effect or chaotropic effect.

### Example 7 Effect of the Presence of Both Chaotropic Reagent and Reducing Agent

Under the low-concentration reducing agent usage conditions, the chaotropic reagent concentration dependency of the detection of Adalimumab by the nSMOL method was studied. More specifically, the nSMOL method was conducted similarly as in Example 1 but using urea in the range of 0 to 3 M as a chaotropic reagent and TCEP in the range of 0.01 to 0.2 mM as a reducing agent (at pH 8.5).

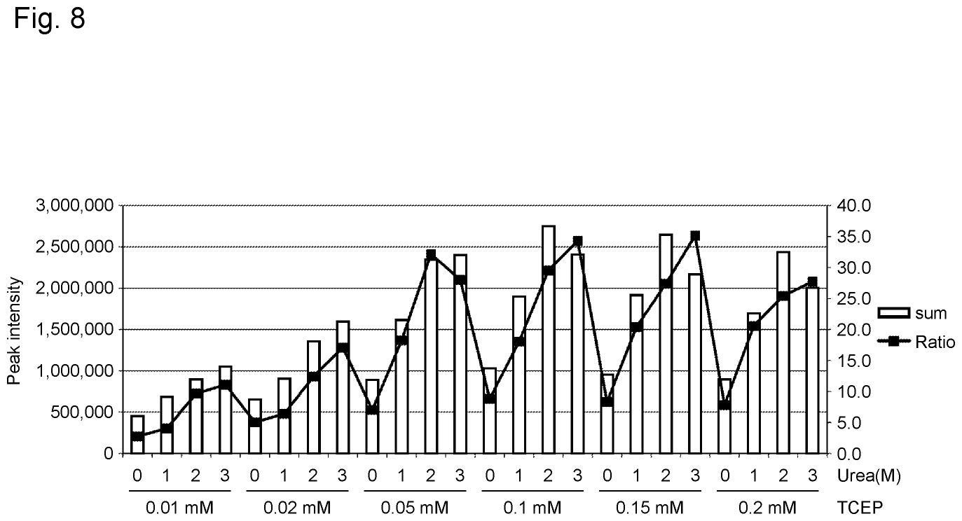

As a result, as illustrated in FIG. 8, it was confirmed that the yield was lowered with 3 M urea depending on the reducing agent concentration. On the other hand, it was found that the added ISTD was increased excessively with respect to the free peptide. From these results, it was considered that the use of 2M urea as a chaotropic reagent and TCEP in the range of 0.1 to 0.2 mM as a reducing agent was optimum.

### Example 8 Effect of Improving Adalimumab Detection Sensitivity

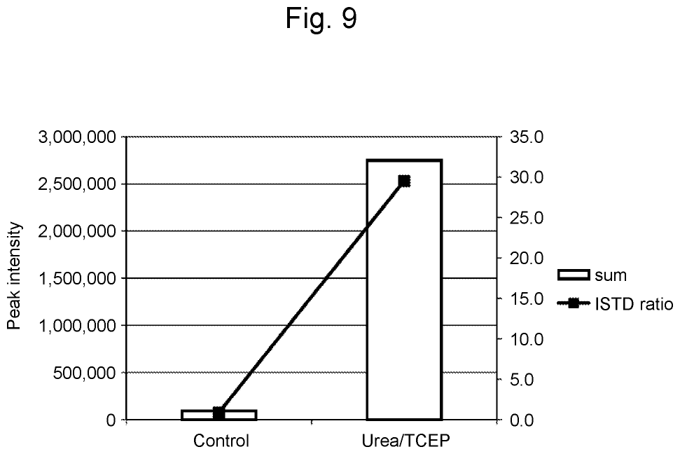

Considering the results in Examples above and various results studied apart from the Examples, comparison of peak intensities in the Adalimumab detection by the nSMOL method was made, comparing a case where the reaction was conducted at pH 8.5 with 2 M urea as a chaotropic reagent and 0.2 mM TCEP as a reducing agent and a case where the reaction was made at pH 8 in the absence of the chaotropic reagent and the reducing agent, while the cases were identical with each other for other conditions. As illustrated in FIG. 9, the results demonstrated that the method according to the present invention (Urea/TCEP) achieved such a significant improvement effect, attaining a value higher than the conventional method (control) by about 30 times.

### Example 9 Creation of Calibration Curve

Using the condition where the improvement in sensitivity was confirmed in Example 8 (2 M urea, 0.2 mM TCEP, pH 8.5), samples containing Adalimumab in the concentration range of 2 to 250 μg/mL were analyzed by the nSMOL method.

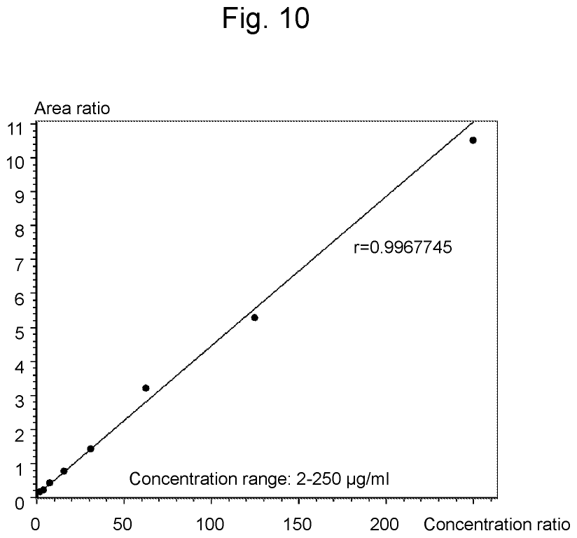

As a result, as illustrated in FIG. 10, a substantially linear quantification results in proportion with the concentration was obtained (r=0.9967745), which satisfies the analysis guideline standard. That is, it was demonstrated that the method according to the present invention is a highly reliable analysis method.

### Example 10 Study of Reducing Agent Concentration Dependency Using a Plurality of Antibodies

Various antibodies were studied similarly as in Adalimumab. For each of Trastuzumab (Chugai Pharmaceutical Co., Ltd.), Cetuximab (Bristol-Myers Squibb Company). Rituximab (Zenyaku Kogyo Company, Limited), and Nivolumab (ONO PHARMACEUTICAL CO., LTD.), the signature peptides shown in Table 3 were selected based on the amino acid sequence information etc., the detection was conducted in the presence of 0.1 mM, 0.2 mM, and 0.5 mM TCEP (2M urea, pH 8.5), and detection results were compared.

TABLE 2

| Antibody | Signature peptide | SEQ ID NO. |
|---|---|---|
| Trastuzumab | IYPTNGYTR | 4 |
| Cetuximab | SQVFFK | 5 |
| Rituximab | GLEWIGAIYPGNGDTSYNQK | 6 |
| Nivolumab | ASGITFSNSGMHWVR | 7 |

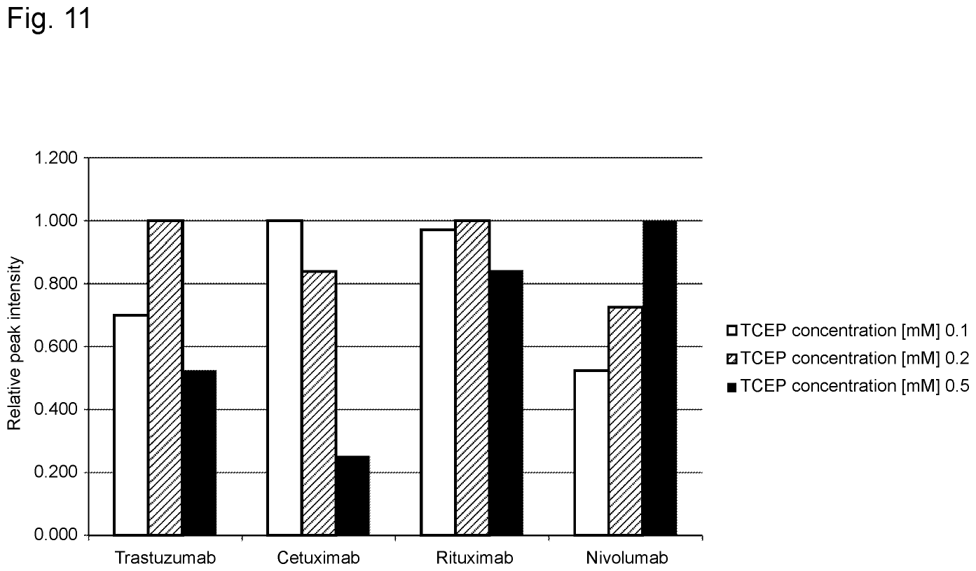

As a result, as illustrated in FIG. 11, it was demonstrated that different antibodies have different TCEP concentration conditions providing the highest peak intensity. As the general conditions for the detection for the four kinds of antibodies that were studied for Adalimumab, in this Example, and Trastuzumab that have been used as reference conditions herein, TCEP concentration of 0.2 mM is considered as being suitable.

### Example 11 Effect of Improving Detection Sensitivity for a Plurality of Antibodies

Using the TCEP concentration of 0.2 mM studied in Example 10, peak intensities of the signature peptides were compared between the control in which the nSMOL method was conducted at pH 8 without the chaotropic reagent and the reducing agent and the case where the nSMOL method was conducted at pH 8.5 with 2M urea and 0.2 mM TCEP for Trastuzumab, Cetuximab, Rituximab, and Nivolumab.

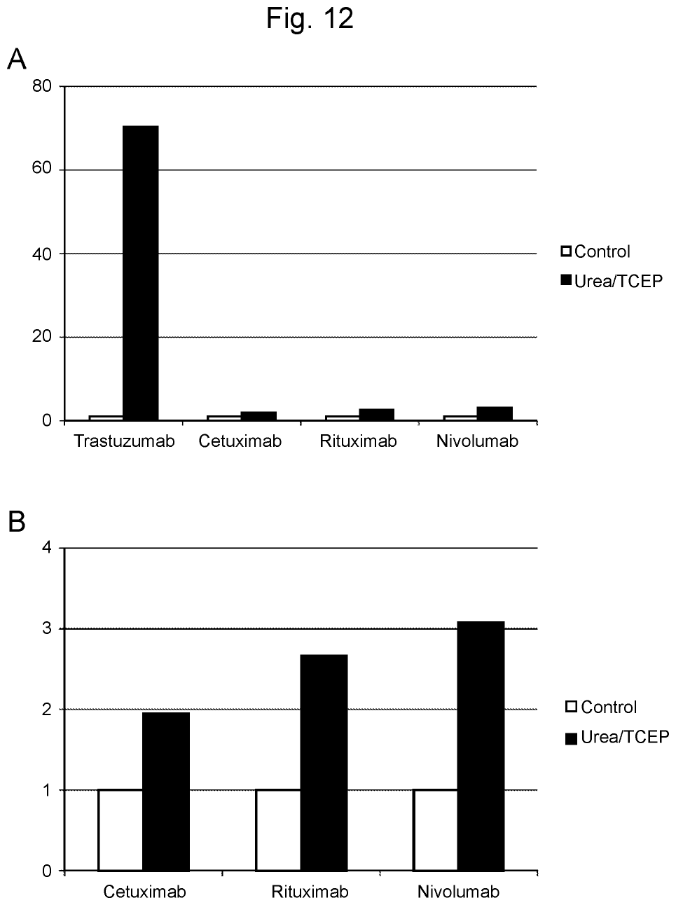

FIG. 12 illustrates relative peak intensities for Trastuzumab, Cetuximab, Rituximab, and Nivolumab, where the peak intensity of the control is 1. For any of these antibodies, a sensitivity improvement effect was clearly observed in the presence of TCEP. Especially for the detection of Trastuzumab, a significant sensitivity increase exceeding 60 times was observed. For Cetuximab, Rituximab, and Nivolumab, sensitivity increases of about 2 to 3 times were observed.

### Example 12 Expansion of the Calibration Curve Range for Trastuzumab Detection

Using the conditions with 2M urea, 0.2 mM TCEP, and pH 8.5, which achieved a significant sensitivity improvement in Example 11 above, samples containing Trastuzumab in the concentration ranges of 2 to 250 μg/mL were analyzed by the nSMOL method. The measurement was conducted with the peptide of SEQ ID NO: 4 as a signature peptide.

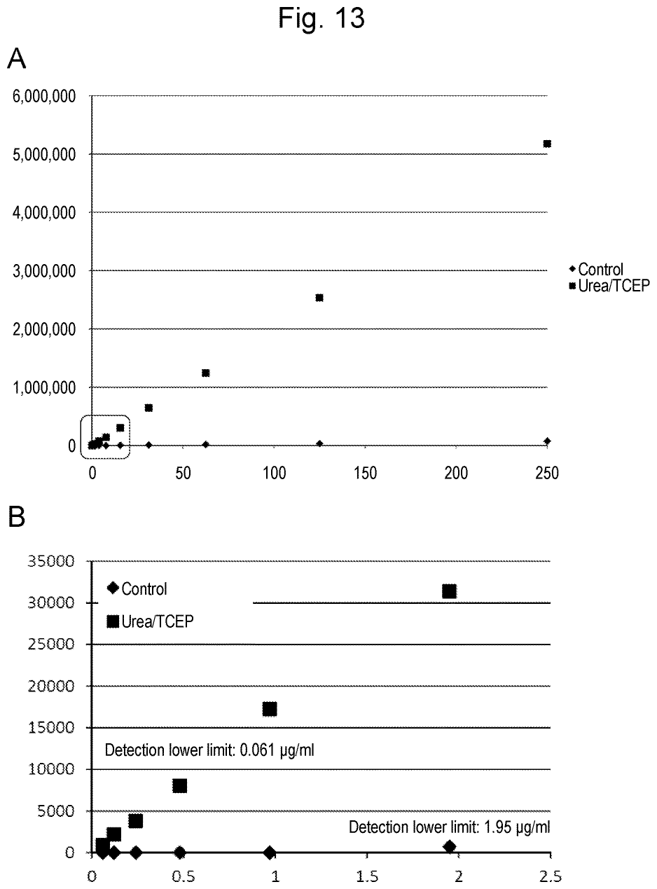

As a result, as illustrated in FIG. 13, sensitivity improvement effects by the method according to the present invention were observed in any concentration. Moreover, in case where the detection was conducted at pH 8 without the reducing agent and the chaotropic reagent, the reliable detection lower limit was 1.95 μg/mL, whereas the detection lower limit according to present invention was 0.061 μg/mL. This demonstrated that the method according to the present invention could not only increase the sensitivity but also detect a significantly low concentration of antibodies.

The results illustrated in FIG. 13 demonstrates that the method of the present invention can detect a much lower antibody concentration with high reliability, based on the comparison with the calibration curve made according to the reaction without the reducing agent and the chaotropic reagent.

### INDUSTRIAL APPLICABILITY

The present invention improves the protocol of the nSMOL method, improving the versatility of the detection method for the monoclonal antibodies using mass spectrometry. Especially for pharmacokinetic studies and therapeutic drug monitoring studies, the present invention makes the nSMOL method applicable for a wide range of various antibody drugs including antibodies for which the conventional method would possibly produce low detection results.

All publications, patents and patent applications cited in the present specification are incorporated herein by reference in their entirety.

1. A method for improving a detection sensitivity in a detection method for a monoclonal antibody in a sample, the detection method comprising:
(a) a step of capturing the monoclonal antibody in the sample and immobilizing the monoclonal antibody in pores of a porous body;
(b) a step of bringing the porous body in which the monoclonal antibody is immobilized with nanoparticles on which protease is immobilized to conduct selective protease digestion of the monoclonal antibody; and
(c) a step of detecting, by a liquid chromatography mass spectrometry (LC-MS), peptide fragments obtained by the selective protease digestion,
wherein the selective protease digestion of step (b) is conducted at pH 8 to 9 in the presence of a chaotropic reagent and a reducing agent.

2. The method according to claim 1, wherein the chaotropic reagent is selected from the group consisting of guanidinium hydrochloride, urea, thiourea, ethylene glycol, and ammonium sulfate.

3. The method according to claim 2, wherein the chaotropic reagent is urea or thiourea in the concentration range of 0.5 to 3 M.

4. The method according to claim 1, wherein the reducing agent is selected from the group consisting of dithiothreitol (DTT), tris(2-carboxyethyl)phosphine (TCEP) or a hydrochloride salt thereof, and tributyl phosphine.

5. The method according to claim 4, wherein the reducing agent is TCEP in the concentration range of 0.1 to 0.5 mM.

6. A use of a chaotropic reagent and a reducing agent for improving detection sensitivity in a detection method of a monoclonal antibody in a sample,
the detection method comprising:
(a) a step of capturing the monoclonal antibody in the sample and immobilizing the monoclonal antibody in pores of a porous body;
(b) a step of bringing the porous body in which the monoclonal antibody is immobilized with nanoparticles on which protease is immobilized to conduct selective protease digestion of the monoclonal antibody; and
(c) a step of detecting, by a liquid chromatography mass spectrometry (LC-MS), peptide fragments obtained by the selective protease digestion.

7. The use according to claim 6, wherein the chaotropic reagent is selected from the group consisting of guanidinium hydrochloride, urea, thiourea, ethylene glycol, and ammonium sulfate.

8. The use according to claim 7, wherein the chaotropic reagent is urea or thiourea in the concentration range of 0.5 to 3 M.

9. The use according to claim 6, wherein the reducing agent is one selected from the group consisting of dithiothreitol (DTT), tris(2-carboxyethyl)phosphine (TCEP) or a hydrochloride salt thereof, and tributyl phosphine.

10. The use according to claim 9, wherein the reducing agent is TCEP in the concentration range of 0.1 to 0.5 mM.


SEQUENCE LISTING (7 sequences)

SEQ ID NO: 1 - protein, 224 aa, Artificial
  Adalimumab H-chain Fab domain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 214 aa, Artificial
  Adalimumab L-chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 10 aa, Artificial
  Adalimumab signature peptide
APYTFGQGTK

SEQ ID NO: 4 - protein, 9 aa, Artificial
  Trastuzumab signature peptide
IYPTNGYTR

SEQ ID NO: 5 - protein, 6 aa, Artificial
  Cetuximab signature peptide
SQVFFK

SEQ ID NO: 6 - protein, 20 aa, Artificial
  Rituximab signature peptide
GLEWIGAIYPGNGDTSYNQK

SEQ ID NO: 7 - protein, 15 aa, Artificial
  Nivolumab signature peptide
ASGITFSNSGMHWVR